(19) United States
(12) Patent Application Publication
(10) Pub. No.: US 2024/0417751 A1
(43) Pub. Date: Dec. 19, 2024

(54) RNA REPLICON FOR IMPROVING GENE EXPRESSION AND USE THEREOF

(71) Applicant: SOUTH CHINA UNIVERSITY OF TECHNOLOGY, Guangzhou (CN)

(72) Inventors: Yuan ZHANG, Guangzhou (CN); Guibin LIN, Guangzhou (CN)

(73) Assignee: SOUTH CHINA UNIVERSITY OF TECHNOLOGY, Guangzhou (CN)

(21) Appl. No.: 18/720,672

(22) PCT Filed: Jan. 29, 2023
(86) PCT No.: PCT/CN2023/073720

§ 371 Date: Jun. 17, 2024

(30) Foreign Application Priority Data
Dec. 17, 2021 (CN) 202111554199.9

(51) Int. Cl. C12N 15/86; A61K 45/06; A61K 48/00; C12N 15/10; C12N 15/70

(52) U.S. Cl. CPC C12N 15/86; A61K 45/06; A61K 48/0041; C12N 15/1031; C12N 15/70; C12N 2770/36141

(57) ABSTRACT

Disclosed in the present invention are an RNA replicon for improving gene expression, and a use thereof. The RNA replicon comprises: 5′ and 3′ untranslated regions; a non-structural protein gene coding region, a subgenomic promoter and a target gene coding region. In the present invention, a PCR site-directed mutagenesis technique is used to introduce the non-structural protein region mutant replicable RNA, which is transfected into mammalian eukaryotic cells by means of Lipofectamine 2000 or nanoparticles, so as to significantly enhance the expression of cytokines and chemokines including GM-CSF, IFN-γ, IL-2, IL-12, IL-15 mediated by downstream subgenomic promoters, and can be applied to treatment of tumors, infectious diseases, autoimmune diseases, hereditary diseases or cardiovascular diseases.

### TECHNICAL FIELD

The present invention belongs to the technical field of genetic engineering, and specifically relates to an RNA replicon for improving gene expression and use thereof.

### RELATED ART

The strategy of introducing a certain or some particular gene(s) into a specific tissue and cell has been widely applied in studies on gene therapy against various diseases. In vivo delivery of genetic materials is generally achieved by means of molecular biology, and a target gene sequence is cloned into a genetic vector; and generally, it is delivered into cells in a form of DNA or RNA encoding the target gene. DNA molecules introduced into cytoplasm via endocytosis further need to span nuclear envelope to complete the delivery and expression of a target gene; and foreign DNA molecules delivered to cell nucleus will be merged into cell genomes, which is prone to inducing tumor formation. Therefore, low delivery efficiency of DNA into cell nucleus, medication safety, and other problems affect the clinical applications of gene therapy with DNA as a carrier. Transcription and translation of messenger RNA molecules (mRNA) only occur in cytoplasm of a target cell without spanning nuclear envelope, which eliminates the risk of genome integration and enhances the safety of gene therapy. mRNA can be in vitro synthesized in an acellular way and thus allowed to be produced on a large scale fast, which avoids problems, such as complex manufacturing associated with recombinant proteins and viral vectors, thereby accelerating clinical transformation. However, the expression time of mRNA is short, and generally, a protein of interest is degraded after being expressed transiently for 2-3 d. To maintain the therapeutic effect, multiple repeated administration is generally required to effectively regulate the gene expression and effect of gene therapy, which limits its clinical generalization and patient compliance, and increases the treatment cost.

Replicable RNA (repRNA), also called an RNA replicon or self-amplified RNA, is derived from plus-strand or minus-strand RNA virus. The virus can be replicated after infecting host cells. Viral genomes encode some non-structural proteins and structural proteins. RNA replicons used in gene therapy contain non-structural protein genes which encode a replication mechanism of alphavirus RNA; but at least one gene encoding a structural protein of alphavirus is deleted or there exists a gene which fails to encode a structural protein for virus formation, it is regarded as an “incapacitated” virus and thus, cannot produce contagious descendants. An alphavirus replicon includes functional elements (FIG. 1) such as an untranslated region, a non-structural protein gene coding region, a subgenomic promoter, and a target gene coding region.

After repRNA is introduced into cells, repRNA released into cytoplasm can serve as a template to duplicate and synthesize multiple transcripts via an RNA-dependent RNA polymerase, thereby increasing the transcriptional templates, translating and expressing multiple proteins of interest (FIG. 2). Due to shortage of a structural protein, the alphavirus replicon has low intrinsic immunogenicity to a vector per se, and a same repRNA can be repeatedly injected for many times.

### SUMMARY OF INVENTION

The objective of the present invention is to provide an RNA replicon for improving gene expression and use thereof.

The present invention adopts the following technical solution:

In a first aspect of the present invention, provided is an RNA replicon, including a 5′ untranslated region, a 3′ untranslated region, a non-structural protein gene coding region, a subgenomic promoter, and a target gene coding region; any one of mutations (I)-(III) occurs in the non-structural protein gene coding region:

(I) a mutation in at least one of sites selected from G357, G1569, A1572, and C1575 of a non-structural protein 1 as well as T3922 of a non-structural protein 2, and preferably a simultaneous mutation;

(II) a mutation in at least one of sites selected from G357, G1569, A1572, and C1575 of a non-structural protein 1 as well as A3821 and T3922 of a non-structural protein 2, and preferably a simultaneous mutation; and

(III) a mutation including but not limited to at least one of sites G3892 of a non-structural protein 2 and A4714 of a non-structural protein 3, and preferably a simultaneous mutation.

In some embodiments of the present invention, the 5′ untranslated region, the 3′ untranslated region, the non-structural protein gene coding region, and the subgenomic promoter are derived from an alphavirus, a flavivirus, a picornavirus, a paramyxovirus, or a calicivirus. In some preferred embodiments of the present invention, the alphavirus is a Venezuelan equine encephalitis virus, a Sindbis virus, or a Semliki Forest virus; the flavivirus is a Dengue fever virus or a Kunjin virus; the picornavirus is a poliovirus or a human rhinovirus; the paramyxovirus is a caninedistempervirus; and the calicivirus is a feline calicivirus.

In some more preferred embodiments of the present invention, the alphavirus is a Venezuelan equine encephalitis virus.

In some preferred embodiments of the present invention, the RNA replicon includes from the 5′ end to the 3′ end: a 5′ untranslated region, a non-structural protein sequence, a target gene coding sequence, and a 3′ untranslated region.

In some embodiments of the present invention, the RNA replicon further includes a subgenomic promoter; the subgenomic promoter is between the non-structural protein sequence and the target gene coding sequence, and used for regulating translation of the target gene.

In some embodiments of the present invention, the RNA replicon is obtained by in vitro transcription of a DNA-dependent RNA polymerase promoter (T7, T3, SP6) derived from bacteriophage; preferably, the DNA-dependent RNA polymerase promoter is a T7 promoter.

In some embodiments of the present invention, the RNA replicon further contains a 5′-cap and a 3′ poly-A tail, where the 5′-cap structure is added by a vaccinia virus capping system; a 7-methylguanosine cap structure is added at the 5′ end, and S-adenosylmethionine (SAM) serves as a methyl donor for transmethylase; a methyl group is added at 2′-O of the first nucleotide adjacent to the cap structure of the RNA 5′ end; 20-500 bases A are added at the 3′ end of the RNA replicon via E. coli poly (A) polymerase.

In some embodiments of the present invention, the DNA sequence of the non-structural protein region of the RNA replicon is shown in SEQ ID NO:1.

In some embodiments of the present invention, the target gene includes at least one of a tumor-specific or associated antigen, a pathogen-specific or associated antigen, a cytokine or a receptor thereof, a chemokine or a receptor thereof, a growth factor or a receptor thereof, an antibody protein, a cytokine-antibody fusion protein, and an immune checkpoint-associated protein; preferably, the cytokine or the chemokine is a granulocyte-macrophage colony stimulating factor GM-CSF, an interferon-γ (IFN-γ), an interleukin-2 (IL-2), interleukin-12 (IL-12), or interleukin-15 (IL-15). Cytokines can enhance immunoreaction; chemokines can induce adjacent responder cells (e.g., leukocyte) to migrate towards infection sites; they play an important role in genesis and development, and treatment of infections, immunoreactions, inflammations, trauma, septicemia, or cancers.

Wherein repRNA may encode any target gene sequence, e.g., molecular or vaccine antigens for the treatment of diseases.

In a second aspect of the present invention, provided is a vector, including the first aspect of the RNA replicon.

In a third aspect of the present invention, provided is a cell, including the second aspect of the vector.

In some embodiments of the present invention, the recombinant cell is not a novel plant or animal variety.

In a fourth aspect of the present invention, provided is use of the first aspect of the RNA replicon in any of (I)-(V):
(I) delivery of a target gene;
(II) achieving long-acting expression of a target gene;
(III) improving an expression quantity of a target gene;
(IV) gene therapy; and
(V) vaccine research and development.

In a fifth aspect of the present invention, provided is a composition, including the first aspect of the RNA replicon or the second aspect of the vector.

In some embodiments of the present invention, the composition further includes at least one of a medicinal diluent, a medicinal excipient, a medicinal vector, and a medicinal carrier.

In some preferred embodiments of the present invention, the composition may be matched with other agents for combined use; the agents include but are not limited to: monoclonal antibody drugs, bispecific antibody drugs, antibody conjugates, fusion protein drugs, nucleic acid drugs, chemical drugs, blood product drugs, lipid drugs, or traditional Chinese medicine extracts.

In some embodiments of the present invention, the medicinal vector is a transfection reagent, a nonviral vector, a polymeric membrane, a biomimic membrane, a biological membrane, or a viral vector that is based on a cationic lipid and commercially available.

In some embodiments of the present invention, the transfection reagent includes but is not limited to Lipofectamine2000, Lipofectamine3000, Lipofectamine8000, Lipofectamine LTX, Lipofectamine RNAiMAX, Lipofectamine. 3.0.

In some embodiments of the present invention, the nonviral vector includes but is not limited to a cationic polymer, a cationic liposome, an anionic liposome, a micelle, an inorganic nanoparticle, or a microsphere.

In some embodiments of the present invention, the polymeric membrane, the biomimic membrane, or the biological membrane includes but is not limited to cytomembrane, an exosome or an extracellular vesicle.

In some embodiments of the present invention, the viral vector includes but is not limited to an adenovirus vector, a retrovirus, a lentivirus, a herpes virus, or a virus-like particle.

In some embodiments of the present invention, the nanocarrier includes but is not limited to a polycationic peptide, a cationic lipid, an anionic lipid, a neutral lipid, helper lipid, or an amphiphilic compound.

In some embodiments of the present invention, the polycationic peptide is a protamine; the cationic lipid is 1,2-dioleoyl-3-trimethylammonium propane; the helper lipid is cholesterol; the amphiphilic compound is poly(ethylene glycol)-distearoylphosphatidylethanolamine.

In some embodiments of the present invention, the nanocarrier has a particle size of 20-350 nm and an electric charge of −40 mV to 50 mV.

In a sixth aspect of the present invention, provided is a method for expressing a target gene in an organism, including the following steps: administering to the organism the first aspect of the RNA replicon.

In some embodiments of the present invention, the organism is a procaryotic organism or a eukaryotic organism; preferably, a primary cell derived from E. coli, yeast, nematode, fruit fly, mouse, monkey, swine, bovine, canine, rabbit, zebrafish model species, human, mouse, monkey, swine, bovine, canine, rabbit, zebrafish, mammalian cell, and fruit fly or related cell lines.

In some embodiments of the present invention, the mammalian cell includes but is not limited to 293T, B16F10, or 4T1.

In some embodiments of the present invention, the RNA replicon may be administered into cells by transfection, transformation, or infection.

In some embodiments of the present invention, the RNA replicon may be administered into an organism by subcutaneous injection, intracutaneous injection, intramuscular injection, intratumor injection, intravenous injection, intraperitoneal injection, oral administration, intranasal administration, pulmonary drug delivery, or intracranial administration.

The present invention further provides a method for site-specific mutagenesis in a non-structural protein region, specifically simultaneous mutation is performed at G357, G1569, A1572, and C1575 of a non-structural protein 1 and at T3922 of a non-structural protein 2; simultaneous mutation is performed at G357, G1569, A1572, and C1575 of a non-structural protein 1 and at A3821T, T3922 of a non-structural protein 2; and simultaneous mutation is performed at G3892 of a non-structural protein 2 and at A4714 of a non-structural protein 3. In some embodiments of the present invention, a method for the mutation is PCR site-directed mutagenesis.

In some embodiments of the present invention, the mutation primers are as follows:

### G357C Mutation Primers:

G357C F:
(SEQ ID NO: 14)
5′-GAAAATGAAGGAGCTCGCCGCCGTCATGAGCGACCC-3′;
G357C R:
(SEQ ID NO: 15)
5′-GCTCATGACGGCGGCGAGCTCCTTCATTTTCTTGTCC-3′;

Primers for mutation G1569A/A1572C/C1575T:

G1569A/A1572C/C1575T F:
(SEQ ID NO: 16)
5′-GGAGCCCACTCTGGAAGCCGATGTCGACTTGATGTTACAAGAGG-
3′;
G1569A/A1572C/C1575T R:
(SEQ ID NO: 17)
5′-TAACATCAAGTCGACATCGGCTTCCAGAGTGGGCTCCTCAACATC-
3′;

### A3821T Mutation Primers:

A3821T F:
(SEQ ID NO: 10)
5′-CATTGGTGCTATAGCGCGGCTGTTCAAGTTTTCCCGGGTATGCAAA
C-3′;
T7VEESmaI R:
(SEQ ID NO: 11)
5′-GCTTAAGTTAGTTGCGGCCGCCCGGGTCGACTCTAG-3′;

### T3922C Mutation Primers:

T3922C F:
(SEQ ID NO: 4)
5′-GCCCGTACGCACAATCCTTACAAGCTTTCATCAAC-3′;

T3922C R:
(SEQ ID NO: 5)
5′-TGAAAGCTTGTAAGGATTGTGCGTACGGGCCTTG-3′;

### G3892C Mutation Primers:

G3892C F
(SEQ ID NO: 6)
5′-CTGTTTGTATTCATTCGGTACGATCGCAAGGCCCGTAC-3′;
G3892C R
(SEQ ID NO: 7)
5′-CCTTGCGATCGTACCGAATGAATACAAACAGAACTTC-3′;

### A4714G Mutation Primers:

A4714G F
(SEQ ID NO: 8)
5′-TATATCCTCGGAGAAGGCATGAGCAGTATTAGGTCG-3′;
A4714G R
(SEQ ID NO: 9)
5′-TAATACTGCTCATGCCTTCTCCGAGGATATACATGC-3′.

Advantageous effects of the present invention are as follows:

In the non-structural protein region of repRNA, non-structural protein 1 initiates the synthesis of minus strand RNA and participates the capping of 5′ end of viral RNA, and it is essential to the combination of an RNA replicase complex with plasma membrane. Non-structural protein 2 not only adjusts the synthesis of subgnomic RNA, but also serves as RNA helicase and protease for processing multiple proteins. Non-structural protein 3 regulates the interaction between virus and host protein, and participates in the transcription of subgenomes. Site mutation of the non-structural protein region may affect the functions of the non-structural proteins, thus resulting in the change of the expression of a target gene encoded downstream.

To further enhance the expression of the antigen encoded by repRNA and promote the immunoreaction caused by an RNA vaccine, PCR site-directed mutagenesis is utilized in the present invention to make some mutations at specific sites of the non-structural protein region of an alphavirus-derived repRNA; specifically as follows: simultaneous mutation at G357C/G1569A/A1572C/C1575T of a non-structural protein 1 and at T3922C of a non-structural protein 2 introduced into the non-structural protein region, or simultaneous mutation at G357C/G1569A/A1572C/C1575T of a non-structural protein 1 and at A3821T/T3922C of a non-structural protein 2 introduced into the non-structural protein region, which enhances the expression of the target gene encoded downstream of a subgenomic promoter of repRNA; this is probably because these mutations facilitate the stability of the RNA structure or up-regulate the activity of the RNA-dependent RNA polymerase translated in the non-structural protein region. Results of the enzyme-linked immunosorbent assay (ELISA) show that the simultaneous mutation at G357C/G1569A/A1572C/C1575T of the non-structural protein 1 and at T3922C of the non-structural protein 2, or the simultaneous mutation at G357C/G1569A/A1572C/C1575T of the non-structural protein 1 and at A3821T/T3922C of the non-structural protein 2 in the non-structural protein region of repRNA may significantly up-regulate the expression of GM-CSF, IFN-γ, IL-2, IL-12, and IL-15 downstream of the subgenomic promoter, of which the simultaneous mutation at G357C/G1569A/A1572C/C1575T of the non-structural protein 1 and at A3821T/T3922C of the non-structural protein 2 achieves more obvious effect.

Nanoparticles have been proved as vectors for the delivery of nucleic acids, proteins, peptides or drugs, and are applied to the clinic treatment of multiple diseases; but high concentration of drug is toxic and thus prone to causing adverse reaction to organism. In the present invention, the repRNA of a non-structural protein regional mutant (VEE:nsP1GGAC-nsP2T or VEE:nsP1GGAC-nsP2AT) is transfected into a mammalian cell 293T by Lipofectamine2000 or a nanoparticle, which can up-regulate the expression of the target gene mediated by a subgenomic promoter. It indicates that the present invention may decrease the drug dose of the nanoparticle while ensuring the therapeutic effect. Therefore, the present invention is of a great clinical transformation potential and application value. GM-CSF, IFN-γ, IL-2, IL-12, and IL-15 are key molecules to regulate organism immunoreaction and play an important role in the treatment of various diseases. Experimental data of the present invention show that GM-CSF, IFN-γ, IL-2, IL-12, and IL-15 encoded by the repRNA of the non-structural protein regional mutant (VEE:nsP1GGAC-nsP2T or VEE:nsP1GGAC-nsP2AT) have significantly up-regulated expression, indicating that the achievement is of an application value in the clinical treatment of correlated diseases.

In conclusion, according to the present invention, the repRNA of the non-structural protein regional mutant introduced by PCR site-directed mutagenesis is transfected into a mammalian cell by Lipofectamine2000 or a nanoparticle, which can significantly enhance the expression of cytokines or chemokines mediated by the downstream subgenomic promoter thereof, including GM-CSF, IFN-γ, L-2, IL-12 and IL-15. Moreover, the present invention can be applied to the treatment of tumors, infectious diseases, autoimmune diseases, genetic diseases, cardiovascular diseases, or related diseases.

### BRIEF DESCRIPTION OF DRAWINGS

FIG. 1 is a schematic diagram showing a structure of an RNA replicon.

FIG. 2 is a schematic diagram showing replication and gene expression of the RNA replicon in cells.

FIG. 3 shows a chromatogram of a T7-VEE plasmid.

FIG. 4 shows mutation sites in a non-structural protein region of the T7-VEE plasmid.

FIG. 5 shows a sequencing result of a mutation site nsP1 G357C of a plasmid T7-VEE (nsP1GGAC)-GFP.

FIG. 6 shows a sequencing result of a mutation site nsP1 G1569A/A1572C/C1575T of the plasmid T7-VEE (nsP1GGAC)-GFP.

FIG. 7 shows a sequencing result of a mutation site nsP2 T3922C of a plasmid T7-VEE (nsP1GGAC-nsP2T)-GFP.

FIG. 8 shows a sequencing result of a mutation nsP2 G3892C of a plasmid T7-VEE (nsP1GGAC-nsP2GT-nsP3A)-GFP.

FIG. 9 shows a sequencing result of a mutation site nsP3 A4714G of a plasmid T7-VEE (nsP1GGAC-nsP2GT-nsP3A)-GFP.

FIG. 10 shows a sequencing result of a mutation site nsP2 G3892C of a plasmid T7-VEE (nsP2G-nsP3A)-GFP.

FIG. 11 shows a sequencing result of a mutation site nsP3 A4714G of the plasmid T7-VEE (nsP2G-nsP3A)-GFP.

FIG. 12 shows a sequencing result of a mutation site nsP2 A3821T of a plasmid T7-VEE (nsP1GGAC-nsP2AT)-GFP.

FIG. 13 shows an ELISA result that repRNA encoding IL-12 and having a wild-type non-structural protein region or a correlated mutation is transfected into a cell 293T by Lipofectamine2000.

FIG. 14 shows an ELISA result that repRNA encoding IL-12 and having a wild-type non-structural protein region or a correlated mutation is transfected into a cell 293T by a nanoparticle.

FIG. 15 shows an ELISA result that repRNA encoding IL-15 and having a wild-type non-structural protein region or a correlated mutation is transfected into a cell 293T by Lipofectamine2000.

FIG. 16 shows an ELISA result that repRNA encoding IL-15 and having a wild-type non-structural protein region or a correlated mutation is transfected into a cell 293T by a nanoparticle.

FIG. 17 shows an ELISA result that repRNA encoding GM-CSF and having a wild-type non-structural protein region or a correlated mutation is transfected into a cell 293T by Lipofectamine2000.

FIG. 18 shows an ELISA result that repRNA encoding GM-CSF and having a wild-type non-structural protein region or a correlated mutation is transfected into a cell 293T by a nanoparticle.

FIG. 19 shows an ELISA result that repRNA encoding IFN-γ and having a wild-type non-structural protein region or a correlated mutation is transfected into a cell 293T by Lipofectamine2000.

FIG. 20 shows an ELISA result that repRNA encoding IFN-γ and having a wild-type non-structural protein region or a correlated mutation is transfected into a cell 293T by a nanoparticle.

FIG. 21 shows an ELISA result that repRNA encoding IL-2 and having a wild-type non-structural protein region or a correlated mutation is transfected into a cell 293T by Lipofectamine2000.

FIG. 22 shows an ELISA result that repRNA encoding IL-2 and having a wild-type non-structural protein region or a correlated mutation is transfected into a cell 293T by a nanoparticle.

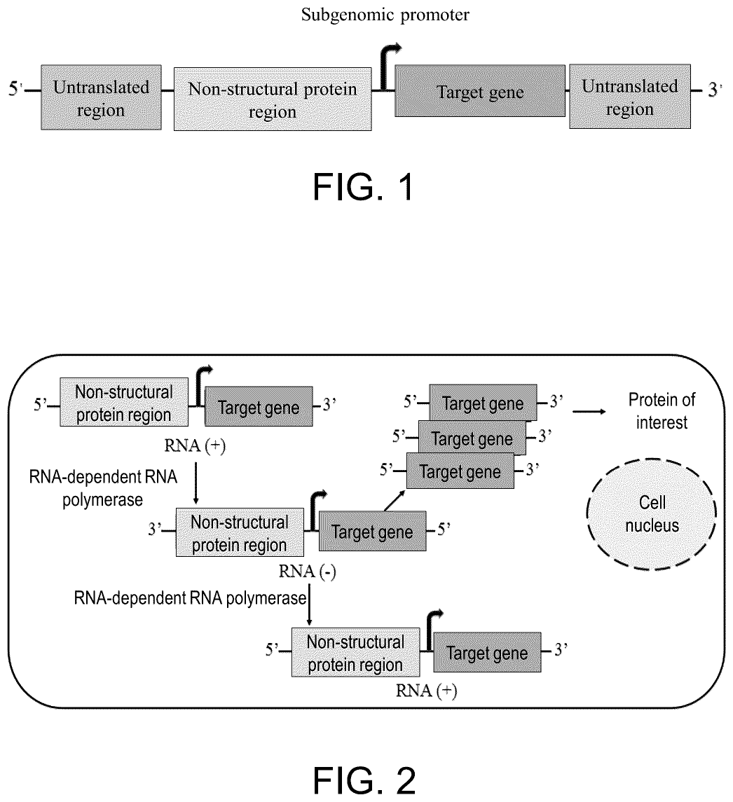

### DESCRIPTION OF EMBODIMENTS

The concept of the present invention and technical effect achieved thereby will be described clearly and completely with reference to the examples below, thus fully understanding the objectives, features and effects of the present invention. Obviously, the examples described are merely a portion of, but are not all the embodiments of the present invention. Based on the examples of the present invention, other examples obtained by those skilled in the art without any inventive effort shall fall within the scope of protection of the present invention.

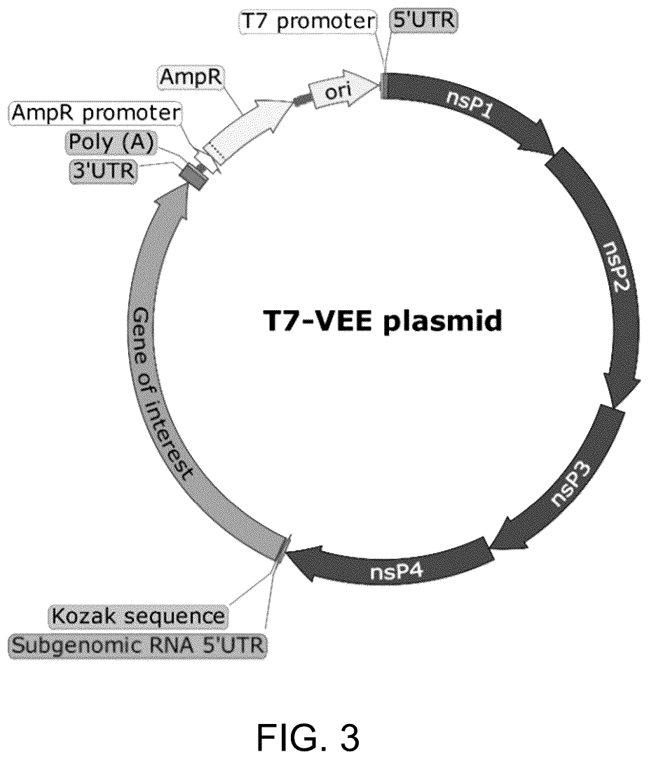

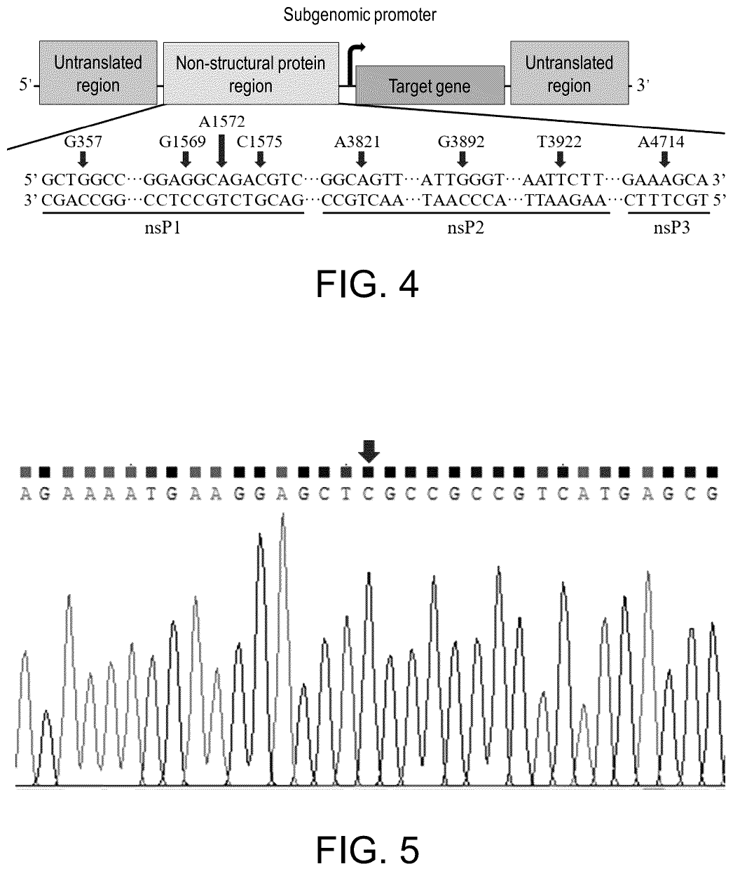

T7-VEE-GFP (Addgene, 58977) with a wild type non-structural protein region (FIG. 3), i.e., a plasmid T7-VEE (WT)-GFP serves as a template, in which the DNA sequence of the non-structural protein region in the RNA replicon is shown in SEQ ID NO:1; sequence of the wild-type plasmid T7-VEE (WT)-GFP is shown in SEQ ID NO:25; a plasmid T7-VEE containing site mutants of the non-structural protein region is thus constructed; the mutation sites are shown in FIG. 4.

### Example 1 Construction of a Mutant nsP1 G357C/G1569A/A1572C/C1575T-nsP2 T3922C

i.e., T7-VEE (nsP1GGAC-nsP2T)-GFP:

### 1) Restriction Enzyme Cutting Sites and Primers of the Vector T7-VEE:

T7VEEBglII F
(SEQ ID NO: 2)
5′-AAAAGCGCAGTCACCAAAAAAGATCTAGTGGTGAGCGCC-3′;
T7VEENdeI R
(SEQ ID NO: 3)
5′-ATCGATGCTGAGGGCGCGCCCATATGCTAGAC-3′;

### G357C Mutation Primers:

G357C F
(SEQ ID NO: 14)
5′-GAAAATGAAGGAGCTCGCCGCCGTCATGAGCGACCC-3′;
G357C R
(SEQ ID NO: 15)
5′-GCTCATGACGGCGGCGAGCTCCTTCATTTTCTTGTCC-3′;

### Primers for Mutation G1569A/A1572C/C1575T:

G1569A/A1572C/C1575T F
(SEQ ID NO: 16)
5′-GGAGCCCACTCTGGAAGCCGATGTCGACTTGATGTTACAAGAGG-
3′;
G1569A/A1572C/C1575T R
(SEQ ID NO: 17)
5′-TAACATCAAGTCGACATCGGCTTCCAGAGTGGGCTCCTCAACATC-
3′;

### T3922C Mutation Primers:

T3922C F
(SEQ ID NO: 4)
5′-GCCCGTACGCACAATCCTTACAAGCTTTCATCAAC-3′;
T3922C R
(SEQ ID NO: 5)
5′-TGAAAGCTTGTAAGGATTGTGCGTACGGGCCTTG-3′;

PCR amplification system: 12.3 μL of ultrapure water, 4 μL of 5×HF buffer solution, 0.4 μL of 10 mM dNTP, 1 μL of primer F, 1 μL of primer R, 0.5 μL of plasmid T7-VEE (WT)-GFP, 0.6 μL of dimethyl sulfoxide, and 0.2 μL of DNA polymerase;
amplification procedure: 30 s at 98° C.; 10 s at 98° C., 10 s at 55° C., 30 s/kb at 72° C., 30 cycles; 8 min at 72° C.

T7-VEE (nsP1GGAC)-GFP served as a template to PCR amplify the forward fragment (1748 bp) containing a mutation T3922C using primers T7VEEBglIIF and T3922CR, and to PCR amplify the reverse fragment (3646 bp) containing a mutation T3922C using primers T3922CF and T7VEENdeIR; the amplified product was subjected to agarose gel electrophoresis, and gel was recovered.

2) The plasmid vector T7-VEE (nsP1GGAC)-GFP was digested by restriction enzymes BgIII, NdeI and XhoI; the restriction enzyme cutting system: 3 μL of 10× buffer solution, 24 μL of plasmid T7-VEE (nsP1GGAC)-GFP, 1 μL of BglII, 1 μL of NdeI, and 1 μL of XhoI.

After reaction for 2 h at 37° C., the product was subjected to agarose gel electrophoresis, and gel was recovered to obtain fragments of 6212 bp.

3) Homologous recombination procedure and the specific reaction system: forward fragment containing a mutation T3922C: 0.01×1748 bp=17.48 ng; reverse fragment containing a mutation T3922C: 0.01×3646 bp=36.46 ng; plasmid vector T7-VEE (nsP1GGAC)-GFP digested by restriction enzymes BglII, NdeI and XhoI: 0.01×6212 bp=62.12 ng; 2× clonExpression Mix: a sum of volumes of the above DNA fragments and the plasmid vector.

After reaction for 15 min at 50° C., the product was immediately put on ice and subjected to standing for 5 min.

4) Transformation: the recombinant product was added to E. coli competent cells, standing for 25 min on ice, 45 s later at 42° C., the product was immediately put on ice for 5 min with the addition of 750 μL of an antibiotic-free LB medium, and subjected to shake culture at 37° C. and 200 rpm for 1 h, and centrifuged for 5 min at 3500 rpm; 600 μL supernatant was discarded, and the remaining liquid was mixed well and coated on an LB plate containing ampicillin, and subjected to inverted culture in a 37° C. incubator over the night.

5) Monoclonal colonies were picked out and identified by restriction analysis of MluI and EcoRI; the enzyme digestion reaction system was as follows: 7.8 μL of ultrapure water, 1 μL of 10× buffer solution, 1 μL of plasmid T7-VEE (WT)-GFP, 0.1 μL of MluI, and 0.1 μL of BglII.

After reaction for 1 h at 37° C., the digested product was subjected to agarose gel electrophoresis, and the plasmid identified correct was sequenced; the sequencing result of the T3922C mutation is shown in FIG. 7.

### Example 2 Construction of a Mutant nsP2 G3892C-nsP3 A4714G

i.e., T7-VEE (nsP2G-nsP3A)-GFP:

1) restriction enzyme cutting sites and primers of the vector T7-VEE: as shown in Example 1:

### G3892C Mutation Primers:

G3892C F
(SEQ ID NO: 6)
5′-CTGTTTGTATTCATTCGGTACGATCGCAAGGCCCGTAC-3′;
G3892C R
(SEQ ID NO: 7)
5′-CCTTGCGATCGTACCGAATGAATACAAACAGAACTTC-3′;

### A4714G Mutation Primers:

A4714G F
(SEQ ID NO: 8)
5′-TATATCCTCGGAGAAGGCATGAGCAGTATTAGGTCG-3′;
A4714G R
(SEQ ID NO: 9)
5′-TAATACTGCTCATGCCTTCTCCGAGGATATACATGC-3′.

The PCR amplification system was the same as that in Example 1; T7-VEE (WT)-GFP served as a template to PCR amplify the forward fragment (1714 bp) containing a mutation G3892C using primers T7VEEBglIIF and G3892CR, and to PCR amplify the intermediate fragment (853 bp) containing mutations G3892C/T3922C and A4714GR using primers G3892CF and A4714GR, and to PCR amplify the reverse fragment (2850 bp) containing a mutation A4714G using primers A4714GF and T7VEENdeIR; the product was subjected to agarose gel electrophoresis, and the gel was recovered.

2) Plasmid vector T7-VEE (WT)-GFP was digested by restriction enzymes BgIII, NdeI and XhoI, and the restriction enzyme system was the same as that in Example 1.

After reaction for 2 h at 37° C., the product was subjected to agarose gel electrophoresis, and gel was recovered to obtain fragments of 6212 bp.

3) Homologous recombination procedure and the reaction system: 17.14 ng of the forward fragment containing a mutation G3892C; 8.53 ng of the intermediate fragment containing mutations G3892C and A4714G, 28.5 ng of the reverse fragment containing a mutation A4714G; 62.12 ng of the plasmid vector T7-VEE (WT)-GFP digested by restriction enzymes BglII, NdeI and XhoI, and 2× clonExpression Mix: a sum of volumes of the above DNA fragments and the plasmid vector.

After reaction for 15 min at 50° C., the product was immediately put on ice and subjected to standing for 5 min.

4) Transformation: the recombinant product was added to E. coli competent cells, standing for 25 min on ice, 45 s later at 42° C., the product was immediately put on ice for 5 min with the addition of 750 μL of an antibiotic-free LB medium, and subjected to shake culture at 37° C. and 200 rpm for 1 h, and centrifuged for 5 min at 3500 rpm; 600 μL supernatant was discarded, and the remaining liquid was mixed well and coated on an LB plate containing ampicillin, and subjected to inverted culture in a 37° C. incubator over the night.

5) Monoclonal colonies were picked out and identified by restriction analysis of BglII and XhoI; the enzyme digestion reaction system was as follows: 7.8 μL of ultrapure water, 1 μL of 10× buffer solution, 1 μL of plasmid, 0.1 μL of BglII, and 0.1 μL of XhoI.

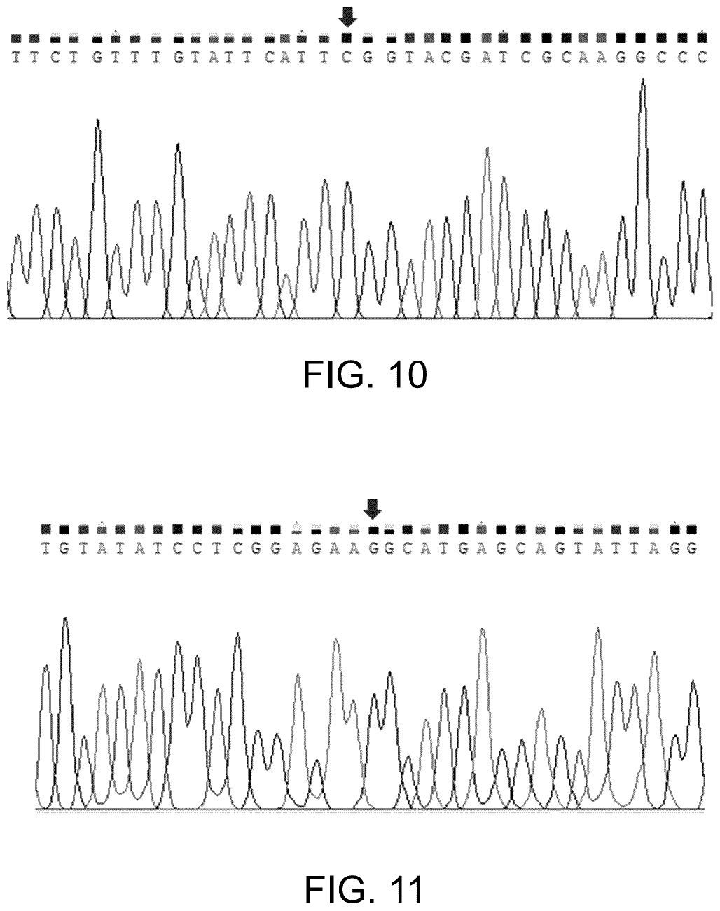

After reaction for 1 h at 37° C., the digested product was subjected to agarose gel electrophoresis, and the plasmid identified correct was sequenced; the sequencing result of the G3892C mutation is shown in FIG. 10, and the sequencing result of the A4714G mutation is shown in FIG. 11.

### Example 3 Construction of a Mutant nsP1 G357C/G1569A/A1572C/C1575T-nsP2 A3821T/T3922C

i.e., T7-VEE (nsP1GGAC-nsP2AT)-GFP:

### A3821T Mutation Primers:

A3821T F
(SEQ ID NO: 11)
5′-CATTGGTGCTATAGCGCGGCTGTTCAAGTTTTCCCGGGTATGCAAA
C-3′;
T7VEESmaI R
(SEQ ID NO: 11)
5′-GCTTAAGTTAGTTGCGGCCGCCCGGGTCGACTCTAG-3′.

The PCR amplification system was the same as that in Example 1; T7-VEE (nsP1GGAC-nsP2T)-GFP served as a template to PCR amplify the DNA fragment (4460 bp) containing a mutation A3821T using primers A3821TF and T7VEESmaIR; the product was subjected to agarose gel electrophoresis, and the gel was recovered.

2) The plasmid vector T7-VEE (nsP1GGAC-nsP2T)-GFP was digested by restriction enzyme SmaI; the restriction enzyme cutting system: 3 μL of 10× buffer solution, 26 μL of T7-VEE (nsP1GGAC-nsP2T)-GFP, and 1 μL of SmaI.

After reaction for 2 h at 37° C., the product was subjected to agarose gel electrophoresis, and gel was recovered to obtain fragments of 7062 bp.

3) Homologous recombination and the reaction system: 44.6 ng of the PCR amplified fragment containing a mutation A3821T; 70.62 ng of the plasmid vector T7-VEE (nsP1GGAC-nsP2T)-GFP digested by the restriction enzyme SmaI, and 2× clonExpression Mix: a sum of volumes of the above DNA fragments and the plasmid vector.

After reaction for 15 min at 50° C., the product was immediately put on ice and subjected to standing for 5 min.

4) Transformation: the recombinant product was added to E. coli competent cells, standing for 25 min on ice, 45 s later at 42° C., the product was immediately put on ice for 5 min with the addition of 750 μL of an antibiotic-free LB medium, and subjected to shake culture at 37° C. and 200 rpm for 1 h, and centrifuged for 5 min at 3500 rpm; 600 μL supernatant was discarded, and the remaining liquid was mixed well and coated on an LB plate containing ampicillin, and subjected to inverted culture in a 37° C. incubator over the night.

5) Monoclonal colonies were picked out and identified by restriction analysis of SmaI; the enzyme digestion reaction system was as follows: 7.9 μL of ultrapure water, 1 μL of 10× buffer solution, 1 μL of plasmid, and 0.1 μL of SmaI.

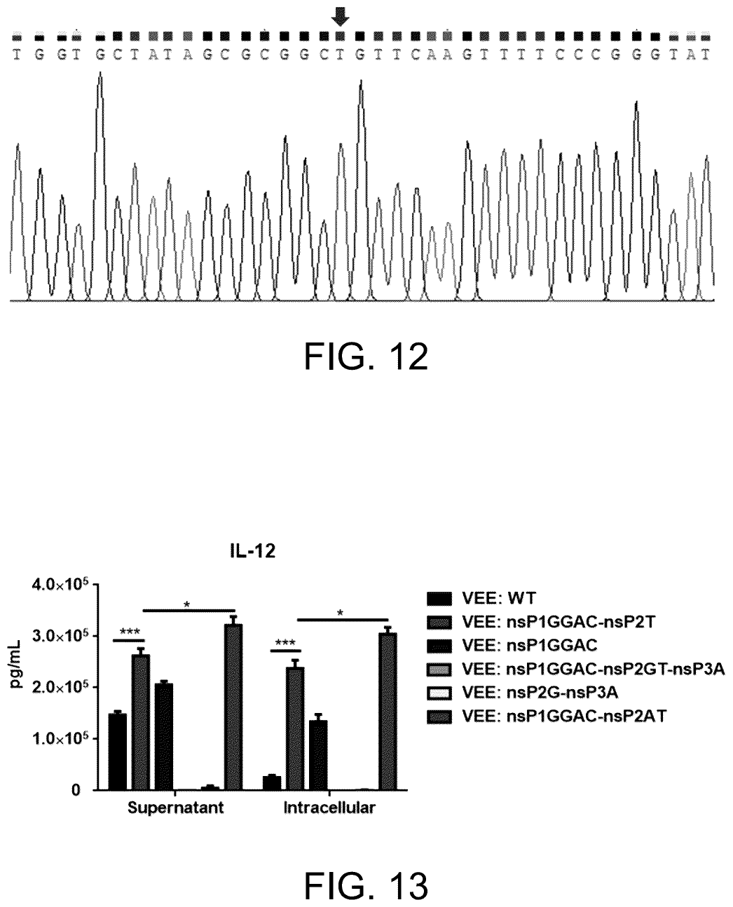

After reaction for 1 h at 37° C., the digested product was subjected to agarose gel electrophoresis, and the plasmid identified correct was sequenced; the sequencing result of the A3821T mutation is shown in FIG. 12.

### Comparative Example 1 Construction of a Mutant nsP1 G357C/G1569A/A1572C/C1575T

i.e., T7-VEE (nsP1GGAC)-GFP:

1) restriction enzyme cutting sites and primers of the vector T7-VEE:

### Restriction Enzyme Cutting Sites and Primers of the Vector T7-VEE:

T7VEEMluI F
(SEQ ID NO: 12)
5′-AAAAAAAAAAAAAAAAAAAACGCGTCGAGGGGAATTAATTCTTGAA
GACG-3′;
T7VEEBglII R
(SEQ ID NO: 13)
5′-CTTTCTTGGCGCTCACCACTAGATCTTTTTTGGTGACTGCGCTTTT
AATG-3′;

Primers for mutation G357C and primers for mutations G1569A/A1572C/C1575T were the same as those in Example 1.

The PCR amplification system was the same as that in Example 1; T7-VEE (WT)-GFP served as a template to PCR amplify the forward fragment (2227 bp) containing a mutation G357C using primers T7VEEMluI F and G357CR, and to PCR amplify the intermediate fragment (1258 bp) containing mutations G357C and G1569A/A1572C/C1575T using primers G357C F and G1569A/A1572C/C1575T R, and to PCR amplify the reverse fragment (687 bp) containing a mutation G1569A/A1572C/C1575T using primers G1569A/A1572C/C1575T F and T7VEEBglII R; the product was subjected to agarose gel electrophoresis, and the gel was recovered.

2) The plasmid vector T7-VEE (WT)-GFP was digested by restriction enzymes MluI and BglII; the restriction enzyme cutting system: 3 μL of 10× buffer solution, 25 μL of plasmid T7-VEE (WT)-GFP, 1 μL of MluI, and 1 μL of BglII.

After reaction for 2 h at 37° C., the product was subjected to agarose gel electrophoresis, and gel was recovered to obtain fragments of 7438 bp.

3) Homologous recombination and the reaction system: 22.27 ng of the forward fragment containing a mutation G357C; 12.58 ng of the intermediate fragment containing mutations G357C and G1569A/A1572C/C1575T, 6.87 ng of the reverse fragment containing a mutation G1569A/A1572C/C1575T; 74.38 ng of the plasmid vector T7-VEE (WT)-GFP digested by restriction enzymes MluI and BglII, and 2× clonExpression Mix: a sum of volumes of the above DNA fragments and the plasmid vector.

After reaction for 15 min at 50° C., the product was immediately put on ice and subjected to standing for 5 min.

4) Transformation: the recombinant product was added to E. coli competent cells, standing for 25 min on ice, 45 s later at 42° C., the product was immediately put on ice for 5 min with the addition of 750 μL of an antibiotic-free LB medium, and subjected to shake culture at 37° C. and 200 rpm for 1 h, and centrifuged for 5 min at 3500 rpm; 600 μL supernatant was discarded, and the remaining liquid was mixed well and coated on an LB plate containing ampicillin, and subjected to inverted culture in a 37° C. incubator over the night.

5) Monoclonal colonies were picked out and identified by restriction analysis of MluI and EcoRI; the enzyme digestion reaction system was as follows: 7.8 μL of ultrapure water, 1 μL of 10× buffer solution, 1 μL of plasmid, 0.1 μL of MluI, and 0.1 μL of EcoRI.

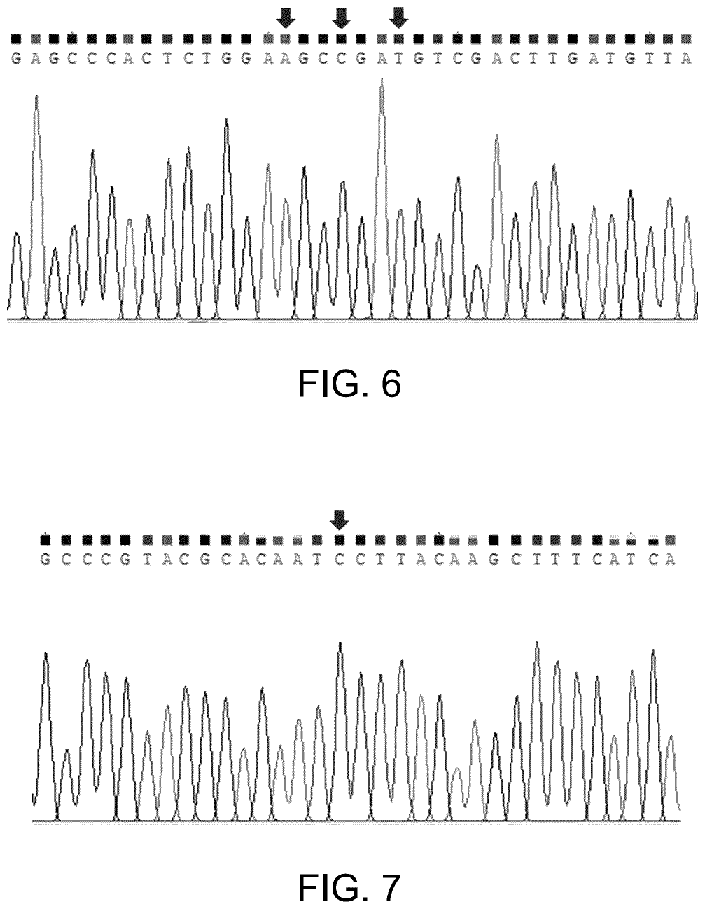

After reaction for 1 h at 37° C., the digested product was subjected to agarose gel electrophoresis, and the plasmid identified correct was sequenced; the sequencing result of the G357C mutation is shown in FIG. 5, and the sequencing result of the G1569A/A1572C/C1575T mutation is shown in FIG. 6.

Comparative Example 2 Construction of a mutant nsP1 G357C/G1569A/A1572C/C1575T-nsP2 G3892C/T3922C-nsP3 A4714G

i.e., T7-VEE (nsP1GGAC-nsP2GT-nsP3A)-GFP:

1) restriction enzyme cutting sites and primers of the vector T7-VEE: as shown in Example 1:

### Primers for Mutation G3892C/T3922C:

G3892C/T3922C F
(SEQ ID NO: 18)
5′-GTTCTGTTTGTATTCATTCGGTACGATCGCAAGGCCCGTACGCACA
ATCCTTACAAGCTTTCATCAAC-3′;
G3892C/T3922C R
(SEQ ID NO: 19)
5′-TTGATGAAAGCTTGTAAGGATTGTGCGTACGGGCCTTGCGATCGTA
CCGAATGAATACAAACAGAAC-3′;

Primers for mutation A4714G were the same as those in Example 2.

The PCR amplification system was the same as that in Example 1; T7-VEE (nsP1GGAC)-GFP served as a template to PCR amplify the forward fragment (1747 bp) containing a mutation G3892C/T3922C using primers T7VEEBglIIF and G3892C/T3922CR, and to PCR amplify the intermediate fragment (856 bp) containing mutations G3892C/T3922C and A4714G using primers G3892C/T3922CF and A4714GR, and to PCR amplify the reverse fragment (2850 bp) containing a mutation A4714G using primers A4714GF and T7VEENdeIR; the product was subjected to agarose gel electrophoresis, and the gel was recovered.

2) The plasmid vector T7-VEE (nsP1GGAC)-GFP was digested by restriction enzymes BgIII, NdeI and XhoI; restriction enzyme cutting system: 3 μL of 10× buffer solution, 24 μL plasmid T7-VEE (nsP1GGAC)-GFP, 1 μL BglII, 1 μL NdeI, and 1 μL XhoI.

After reaction for 2 h at 37° C., the product was subjected to agarose gel electrophoresis, and gel was recovered to obtain fragments of 6212 bp.

3) Homologous recombination procedure and the reaction system: 17.47 ng of the forward fragment containing a mutation G3892C/T3922C; 8.56 ng of the intermediate fragment containing mutations G3892C/T3922C and A4714G, 28.5 ng of the reverse fragment containing a mutation A4714G; 62.12 ng of the plasmid vector T7-VEE (nsP1GGAC)-GFP digested by restriction enzymes BglII, NdeI and XhoI, 2× clonExpression Mix: a sum of volumes of the above DNA fragments and the plasmid vector.

After reaction for 15 min at 50° C., the product was immediately put on ice and subjected to standing for 5 min.

4) Transformation: the recombinant product was added to E. coli competent cells, standing for 25 min on ice, 45 s later at 42° C., the product was immediately put on ice for 5 min with the addition of 750 μL of an antibiotic-free LB medium, and subjected to shake culture at 37° C. and 200 rpm for 1 h, and centrifuged for 5 min at 3500 rpm; 600 μL supernatant was discarded, and the remaining liquid was mixed well and coated on an LB plate containing ampicillin, and subjected to inverted culture in a 37° C. incubator over the night.

5) Monoclonal colonies were picked out and identified by restriction analysis of BglII and XhoI; the enzyme digestion reaction system was as follows: 7.8 μL of ultrapure water, 1 μL of 10× buffer solution, 1 μL of plasmid, 0.1 μL of BglII, and 0.1 μL of XhoI.

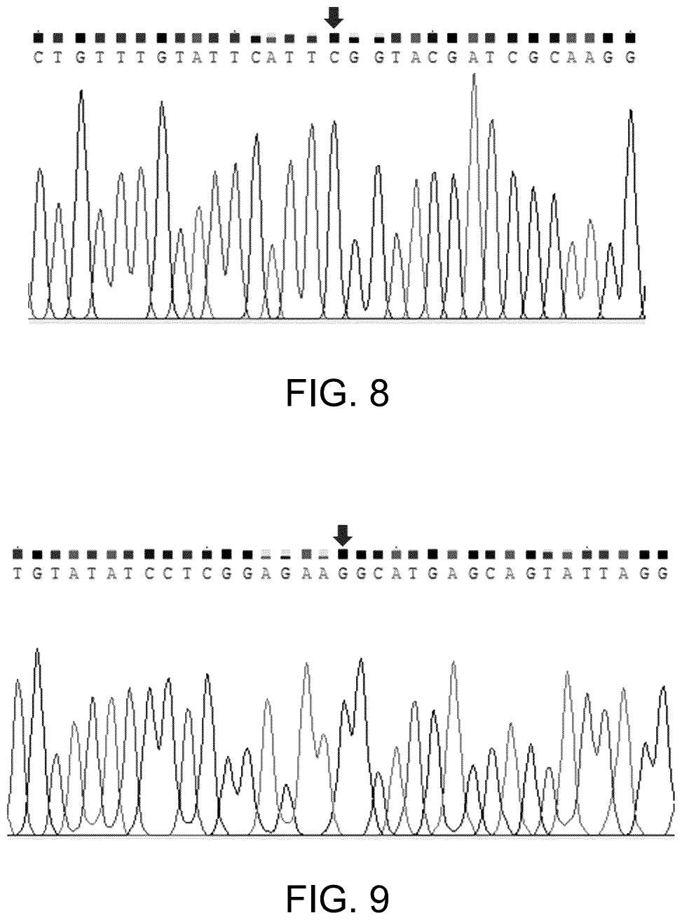

After reaction for 1 h at 37° C., the digested product was subjected to agarose gel electrophoresis, and the plasmid identified correct was sequenced; the sequencing result of the G3892C mutation is shown in FIG. 8, and the sequencing result of the A4714G mutation is shown in FIG. 9.

### Effect Example

### Test Method

Different genes of interest (including cytokines and chemokines) were cloned to a structural protein region.

### 1) PCR Amplification of Genes of Interest

### (i) GM-CSF

Restriction enzyme cutting sites and primers of the vector T7-VEE:

T7VEEGMCSFF
(SEQ ID NO: 20)
5′-GTCTAGTCCGCCAAGTCTAGCATATGGCCACCATGTGGCTGCAG-
3′;
3′UTRR
(SEQ ID NO: 21)
5′-AAAATAAAAATTTTAAGGCGGCATGCCAATCGCCGCGAGTTCTATG
TAAGCAG-3′;

The PCR amplification system was the same as that in Example 1; GM-CSF cDNA (423 bp) was PCR amplified using primers T7VEEGMCSFF and 3′UTRR; the product was subjected to agarose gel electrophoresis, and gel was recovered.

### (ii) IFN-γ

Restriction enzyme cutting sites and primers of the vector T7-VEE:

T7VEEIFNγF
(SEQ ID NO: 22)
5′-GTCTAGTCCGCCAAGTCTAGCATATGGCCACCATGAACGCTACACA
CTGC-3′;

3′UTRR: as shown in SEQ ID NO:21.

The PCR amplification system was the same as that in Example 1; IFN-γ cDNA (46 5 bp) was PCR amplified using primers T7VEEIFNγF and 3′UTRR; the product was subjected to agarose gel electrophoresis, and gel was recovered.

(iii) IL-2

Restriction enzyme cutting sites and primers of the vector T7-VEE:

T7VEED265AF:

(SEQ ID NO: 23)
GTCTAGTCCGCCAAGTCTAGCATATGGCCACCATGGAGACAGACACAC-
3′;

3′UTRR: as shown in SEQ ID NO:21.

The PCR amplification system was the same as that in Example 1; IFN-γ cDNA (561 bp) was PCR amplified using primers T7VEEIFNγF and 3′UTRR; the product was subjected to agarose gel electrophoresis, and gel was recovered.

### (iv) IL-12

Restriction enzyme cutting sites and primers of the vector T7-VEE:

T7VEEIL12F:
(SEQ ID NO: 24)
5′-GTCTAGTCCGCCAAGTCTAGCATATGGCCACC-3′;

3′UTRR: as shown in SEQ ID NO:21.

The PCR amplification system was the same as that in Example 1; IL-12 cDNA (1645 bp) was PCR amplified using primers T7VEEIL12F and 3′UTRR; the product was subjected to agarose gel electrophoresis, and gel was recovered.

### (v) IL-15

Restriction enzyme cutting sites and primers of the vector T7-VEE:

T7VEED265AF: as shown in SEQ ID NO:23;

3′UTRR: as shown in SEQ ID NO:21.

The PCR amplification system was the same as that in Example 1; IL-15 cDNA (753 bp) was PCR amplified using primers T7VEED265AF and 3′UTRR; the product was subjected to agarose gel electrophoresis, and gel was recovered.

2) The above plasmid T7-VEE-GFP was digested by restriction enzymes NdeI and SphI; the reaction system was as follows: 1 μL of ultrapure water, 3 μL of 10× buffer solution, 24 μL of plasmid, 1 μL of NdeI, and 1 μL of SphI.

After reaction for 2 h at 37° C., the product was subjected to agarose gel electrophoresis, and gel was recovered to obtain fragments of 9486 bp.

3) Homologous recombination

(i) GM-CSF, reaction system: 8.46 ng of GM-CSF cDNA; 94.86 ng of the above plasmid T7-VEE-GFP digested by restriction enzymes NdeI and SphI; 2× clonExpression Mix: a sum of volumes of the above DNA fragments and the plasmid vector.

(ii) IFN-γ, reaction system: 9.3 ng of IFN-γ cDNA; 94.86 ng of the above plasmid T7-VEE-GFP digested by restriction enzymes NdeI and SphI, and 2× clonExpression Mix: a sum of volumes of the above DNA fragments and the plasmid vector.

(iii) IL-2, reaction system: 11.22 ng of IL-2 cDNA; 94.86 ng of the above plasmid vector T7-VEE-GFP digested by restriction enzymes NdeI and SphI, and 2× clonExpression Mix: a sum of volumes of the above DNA fragments and the plasmid vector.

(iv) IL-12, reaction system: 32.9 ng of IL-12 cDNA; 94.86 ng of the above plasmid T7-VEE-GFP digested by restriction enzymes NdeI and SphI; and 2× clonExpression Mix: a sum of volumes of the above DNA fragments and the plasmid vector.

(v) IL-15, reaction system: 15.06 ng of IL-15 cDNA; 94.86 ng of the above plasmid T7-VEE-GFP digested by restriction enzymes NdeI and SphI; and 2× clonExpression Mix: a sum of volumes of the above DNA fragments and the plasmid vector.

After reaction for 15 min at 50° C., the product was immediately put on ice and subjected to standing for 5 min.

4) Transformation: the recombinant product was added to E. coli competent cells, standing for 25 min on ice, 45 s later at 42° C., the product was immediately put on ice for 5 min with the addition of 750 μL of an antibiotic-free LB medium, and subjected to shake culture at 37° C. and 200 rpm for 1 h, and centrifuged for 5 min at 3500 rpm; 600 μL supernatant was discarded, and the remaining liquid was mixed well and coated on an LB plate containing ampicillin, and subjected to inverted culture in a 37° C. incubator over the night.

5) Monoclonal colonies were picked out and identified by restriction analysis of MluI and EcoRI; the enzyme digestion reaction system was as follows: 7.8 μL ultrapure water, 1 μL 10× buffer solution, 1 μL plasmid, 0.1 μL MluI, and 0.1 μL EcoRI.

After reaction for 1 h at 37° C., the digested product was subjected to agarose gel electrophoresis, and the plasmid identified correct was sequenced.

6) Linear plasmid T7-VEE was digested by a single restriction enzyme MluI, and the DNA template RNase was removed; reaction system was as follows: 8 μL of 10× buffer solution, 70 μL plasmid, and 2 μL MluI.

7) The purified plasmid T7-VEE was subjected to in vitro transcription with a T7 promoter:

2 μL of 5×T7 transcription buffer solution, 3 μL of rNTPs (25 mM ATP, CTP, GTP, UTP), 3.8 μL (1 μg) of linear DNA template, 1 μL of in vitro transcriptase (T7), and 0.2 μL of Rnasin inhibitor were added to 1.5 mL of an RNase centrifugal tube in order. Reaction was conducted for 3-6 h at 37° C.

8) The T7-VEE plasmid template of the T7 promoter in vitro transcription system was digested by RNase-free DNase and repRNA was purified by lithium chloride.

9) 5′ end of repRNA was capped with methylated guanosine; the repRNA was purified by lithium chloride; the reaction system was as follows: 13.5 μL (10 μg) of uncapped repRNA, 2 μL of 10× capped reaction buffer solution, 1.0 μL of GTP (10 mM), 1.0 μL of S-adenosylmethionine (4 mM), 1.0 μL of vaccinia virus capping enzyme, 1.0 μL of mRNA Cap2 oxymethyltransferase, and 0.5 μL of Rnasin inhibitor. Before capping reaction, repRNA need be heated for 5-25 min at 25-70° C.

10) A poly-A tail (20-500 bases A) was added at the 3′ end of repRNA with capped 5′ end; the repRNA was purified with an RNA purification kit; the reaction system was as follows: 15.5 μL (10 μg) of repRNA with capped 5′ end, 2 μL of 10× poly-A tail-added buffer solution, 1 μL of ATP (10 mM), 1 μL of E. coli poly (A) polymerase, and 0.5 μL of Rnasin inhibitor.

Reaction was conducted for 1 h at 37° C. The repRNA capped with methylated guanosine at 5′ end and capped with poly-A tail at 3′ end was purified using an RNA purification kit.

11) repRNA was transfected into 293T cells using Lipofectamine2000 or a nanoparticle; the expression of the target gene downstream of the subgenomic promoter was determined by ELISA.

11.1 Transfection of repRNA into 293T cells using Lipofectamine2000

About 60% of the 48-well plate were added with 293T cells.

1.5 mL of a centrifugal tube A: 12.5 μL of opti-MEM medium with the addition of 500 ng repRNA.

1.5 mL of a centrifugal tube B: 12.5 μL of opti-MEM medium with the addition of 1 μL Lipofectamine2000.

Tube A was added to Tube B, and mixed well for 5 min at room temperature; and then added to a 293T cell medium.

Cells were cultured for 36 h, the cell medium was collected and cells were lysed.

11.2 Treatment of 293T cells by repRNA enveloped by a nanoparticle

10 μL of nuclease-free water, 500 ng of repRNA, and 375 ng of protamine were mixed for 10-15 min at room temperature, and added with 48.475 nmol of 1,2-dioleoyl-3-trimethylammonium propane/cholesterol, 10-15 min later at room temperature, 2.776 μg of poly(ethylene glycol)-distearoylphosphatidylethanolamine was added, and the obtained product was treated for 12-15 min at 50° C.

11.3 Determination of the expression of the target gene downstream of the subgenomic promoter by ELISA

### Experimental Result

The mutation G357C/G1569A/A1572C/C1575T of a non-structural protein 1, mutation A3821T/G3892C/T3922C of a non-structural protein 2, and mutation A4714G of a non-structural protein 3 were introduced into the non-structural protein region of the in vitro transcription template plasmid of repRNA first by means of PCR site-directed mutagenesis, and these mutations were combined with each other, for example, T7-VEE (nsP1GGAC); T7-VEE (nsP1GGAC-nsP2T); T7-VEE (nsP1GGAC-nsP2AT); T7-VEE (nsP1GGAC-nsP2GT-nsP3A); T7-VEE (nsP2G-nsP3A).

Moreover, different genes of interest were cloned to the structural protein region, mainly including IL-12, IL-15, GM-CSF, IFN-γ, and IL-2; according to the specific expression condition, the major transfection way includes Lipofectamine2000 and nanoparticle.

The ELISA result of the repRNA encoding IL-12 and having a wild-type non-structural protein region or a correlated mutation transfected into a cell 293T by Lipofectamine2000 is shown in FIG. 13; the results show that both mutations VEE nsP1GGAC-nsP2T and VEE nsP1GGAC-nsP2AT up-regulate the intracellular expression and extracellular secretion of IL-12; compared with the mutation VEE nsP1GGAC-nsP2T, the mutation VEE nsP1GGAC-nsP2AT further enhances the intracellular expression and extracellular secretion of IL-12.

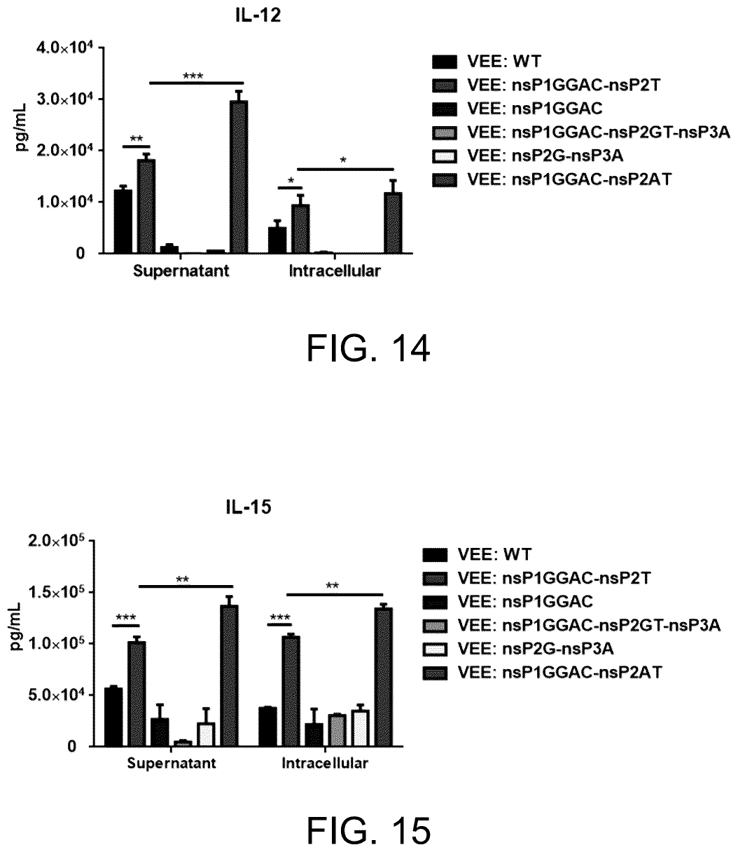

The ELISA result of the repRNA encoding IL-12 and having a wild-type non-structural protein region or a correlated mutation transfected into a cell 293T by a nanoparticle is shown in FIG. 14; the results show that both mutations VEE nsP1GGAC-nsP2T and VEE nsP1GGAC-nsP2AT up-regulate the intracellular expression and extracellular secretion of IL-12; compared with the mutation VEE nsP1GGAC-nsP2T, the mutation VEE nsP1GGAC-nsP2AT further enhances the intracellular expression and extracellular secretion of IL-12.

The ELISA result of the repRNA encoding IL-15 and having a wild-type non-structural protein region or a correlated mutation transfected into a cell 293T by Lipofectamine2000 is shown in FIG. 15; the results show that both mutations VEE nsP1GGAC-nsP2T and VEE nsP1GGAC-nsP2AT up-regulate the intracellular expression and extracellular secretion of IL-15; compared with the mutation VEE nsP1GGAC-nsP2T, the mutation VEE nsP1GGAC-nsP2AT further enhances the intracellular expression and extracellular secretion of IL-15.

The ELISA result of the repRNA encoding IL-15 and having a wild-type non-structural protein region or a correlated mutation transfected into a cell 293T by a nanoparticle is shown in FIG. 16; the results show that both mutations VEE nsP1GGAC-nsP2T and VEE nsP1GGAC-nsP2AT up-regulate the intracellular expression and extracellular secretion of IL-15; compared with the mutation VEE nsP1GGAC-nsP2T, the mutation VEE nsP1GGAC-nsP2AT further enhances the intracellular expression and extracellular secretion of IL-15.

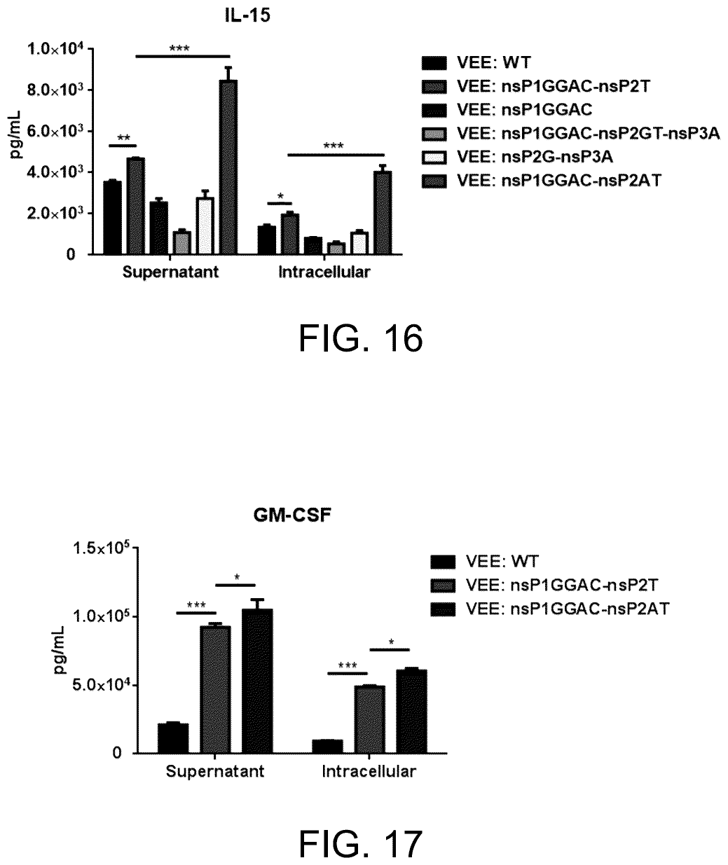

The ELISA result of the repRNA encoding GM-CSF and with a wild-type non-structural protein region or a correlated mutation transfected into a cell 293T by Lipofectamine2000 is shown in FIG. 17; the results show that both mutations VEE nsP1GGAC-nsP2T and VEE nsP1GGAC-nsP2AT up-regulate the intracellular expression and extracellular secretion of GM-CSF; compared with the mutation VEE nsP1GGAC-nsP2T, the mutation VEE nsP1GGAC-nsP2AT further enhances the intracellular expression and extracellular secretion of GM-CSF.

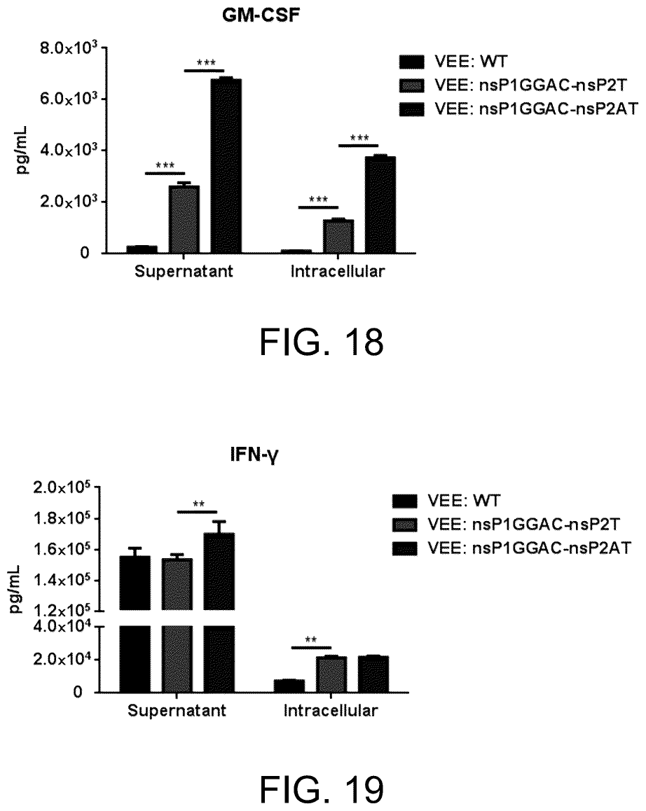

The ELISA result of the repRNA encoding GM-CSF and with a wild-type non-structural protein region or a correlated mutation transfected into a cell 293T by a nanoparticle is shown in FIG. 18; the results show that both mutations VEE nsP1GGAC-nsP2T and VEE nsP1GGAC-nsP2AT up-regulate the intracellular expression and extracellular secretion of GM-CSF; compared with the mutation VEE nsP1GGAC-nsP2T, the mutation VEE nsP1GGAC-nsP2AT further enhances the intracellular expression and extracellular secretion of GM-CSF.

The ELISA result of the repRNA encoding IFN-γ and having a wild-type non-structural protein region or a correlated mutation transfected into a cell 293T by Lipofectamine2000 is shown in FIG. 19; the results show that mutation VEE nsP1GGAC-nsP2AT up-regulates the extracellular secretion of IFN-γ; both mutations VEE nsP1GGAC-nsP2T and VEE nsP1GGAC-nsP2AT up-regulate the intracellular expression of IFN-γ.

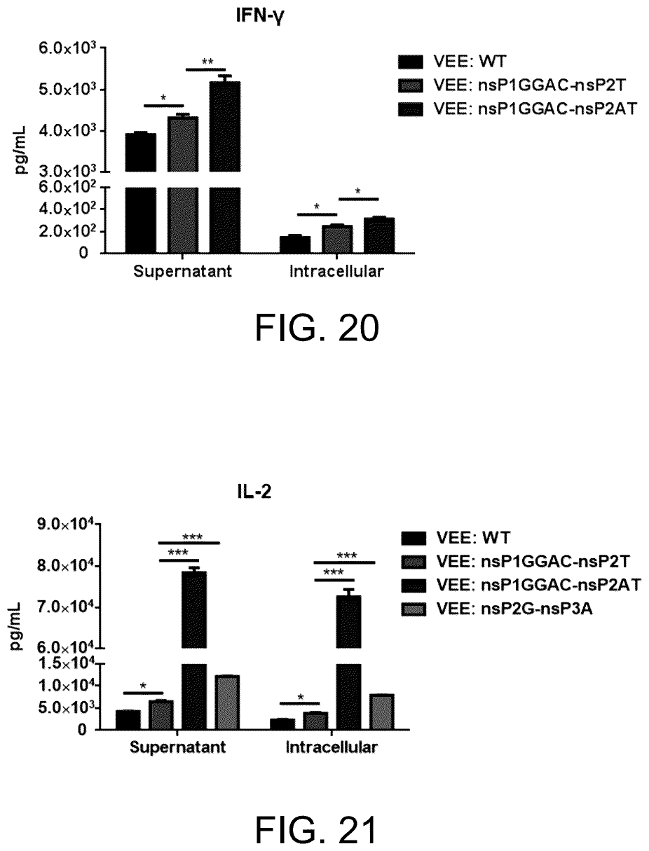

The ELISA result of the repRNA encoding IFN-γ and having a wild-type non-structural protein region or a correlated mutation transfected into a cell 293T by a nanoparticle is shown in FIG. 20; the results show that both mutations VEE nsP1GGAC-nsP2T and VEE nsP1GGAC-nsP2AT up-regulate the intracellular expression and extracellular secretion of IFN-γ; compared with the mutation VEE nsP1GGAC-nsP2T, the mutation VEE nsP1GGAC-nsP2AT further enhances the intracellular expression and extracellular secretion of IFN-γ.

The ELISA result of the repRNA encoding IL-2 and having a wild-type non-structural protein region or a correlated mutation transfected into a cell 293T by Lipofectamine2000 is shown in FIG. 21; the results show that both mutations VEE nsP1GGAC-nsP2T and VEE nsP1GGAC-nsP2AT up-regulate the intracellular expression and extracellular secretion of IL-2; compared with the mutation VEE nsP1GGAC-nsP2T, the mutation VEE nsP1GGAC-nsP2AT further enhances the intracellular expression (the intracellular expression quantity of the IL-2 via the mutation VEE nsP1GGAC-nsP2AT is about 20 times that the IL-2 via the mutation VEE nsP1GGAC-nsP2T) and extracellular secretion (the extracellular secretion volume of the IL-2 via the mutation VEE nsP1GGAC-nsP2AT is about 12 times that the IL-2 via the mutation VEE nsP1GGAC-nsP2T) of the IL-2. The mutation VEE nsP2G-nsP3A up-regulates the intracellular expression and extracellular secretion of IL-2, and its expression level is between the mutation VEE nsP1GGAC-nsP2T and the mutation VEE nsP1GGAC-nsP2AT.

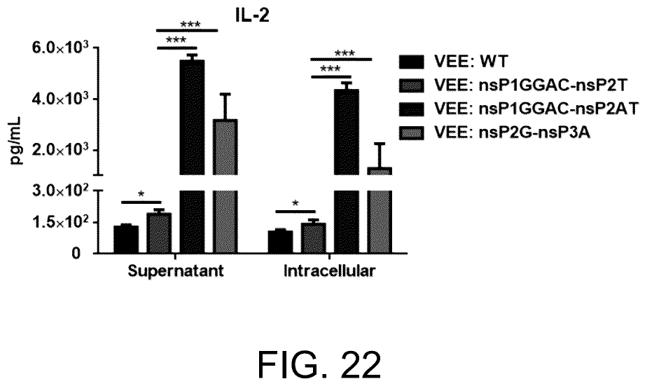

The ELISA result of the repRNA encoding IL-2 and having a wild-type non-structural protein region or a correlated mutation transfected into a cell 293T by a nanoparticle is shown in FIG. 22; the results show that both mutations VEE nsP1GGAC-nsP2T and VEE nsP1GGAC-nsP2AT up-regulate the intracellular expression and extracellular secretion of IL-2; compared with the mutation VEE nsP1GGAC-nsP2T, the mutation VEE nsP1GGAC-nsP2AT further enhances the intracellular expression (the intracellular expression quantity of the IL-2 via the mutation VEE nsP1GGAC-nsP2AT is about 30 times that the IL-2 via the mutation VEE nsP1GGAC-nsP2T) and extracellular secretion (the extracellular secretion volume of the IL-2 via the mutation VEE nsP1GGAC-nsP2AT is about 30 times that the IL-2 via the mutation VEE nsP1GGAC-nsP2T) of the IL-2. The mutation VEE nsP2G-nsP3A up-regulates the intracellular expression and extracellular secretion of IL-2, and its expression level is between the mutation VEE nsP1GGAC-nsP2T and the mutation VEE nsP1GGAC-nsP2AT.

In conclusion, it can be seen that repRNA having a wild-type non-structural protein region or a mutant transcribed in vitro is transfected into a mammalian cell 293T by Lipofectamine2000 or a nanoparticle; and the ELISA results show that both the simultaneous mutation of nsP1 G357C/G1569A/A1572C/C1575T-nsP2 T3922C in the non-structural protein region of repRNA, i.e., VEE (nsP1GGAC-nsP2T) and the mutant nsP1 G357C/G1569A/A1572C/C1575T-nsP2 A3821T/T3922C, i.e., VEE (nsP1GGAC-nsP2AT) may significantly enhance the intracellular expression and extracellular secretion of chemokines or cytokines mediated by the downstream subgenomic promoter thereof, e.g., GM-CSF, IFN-γ, L-2, IL-12 and IL-15. Compared with the mutation VEE nsP1GGAC-nsP2T, the mutation VEE nsP1GGAC-nsP2AT further up-regulates the intracellular expression and extracellular secretion of the above chemokines or cytokines. Moreover, the simultaneous mutation of nsP2 G3892C-nsP3 A4714G in the non-structural protein region of repRNA, i.e., the mutation VEE nsP2G-nsP3A up-regulates the intracellular expression and extracellular secretion of IL-2; and its up-regulation in IL-2 expression ability is between mutations VEE nsP1GGAC-nsP2T and VEE nsP1GGAC-nsP2AT.

The above detailed embodiments are to specify the present invention in detail, but are not construed as limiting the present invention. Those skilled in the art may further make various changes in the premise of not departing from the purpose of the present invention. In addition, examples and features in the examples of the present invention may be combined with each other in case of no conflict.

1. An RNA replicon, comprising in a 5′ to 3′ direction: a 5′ untranslated region, a 3′ untranslated region, a non-structural protein gene coding region, a subgenomic promoter, and a target gene coding region, wherein any one of mutations (I)-(III) occurs in the non-structural protein gene coding region:
(I) a mutation in at least one of sites selected from G357, G1569, A1572, and C1575 of a non-structural protein 1 as well as T3922 of a non-structural protein 2, and preferably a simultaneous mutation;
(II) a mutation in at least one of sites selected from G357, G1569, A1572, and C1575 of a non-structural protein 1 as well as A3821 and T3922 of a non-structural protein 2, and preferably a simultaneous mutation; and
(III) a mutation including but not limited to at least one of sites G3892 of a non-structural protein 2 and A4714 of a non-structural protein 3, and preferably a simultaneous mutation.

2. The RNA replicon according to claim 1, wherein the 5′ untranslated region, the 3′ untranslated region, the non-structural protein gene coding region, and the subgenomic promoter are derived from an alphavirus, a flavivirus, a picornavirus, a paramyxovirus, or a calicivirus;
preferably, the alphavirus is a Venezuelan equine encephalitis virus, a Sindbis virus, or a Semliki Forest virus; the flavivirus is a Dengue fever virus or a Kunjin virus; the picornavirus is a poliovirus or a human rhinovirus; the paramyxovirus is a caninedistempervirus; and the calicivirus is a feline calicivirus, and
more preferably, the alphavirus is a Venezuelan equine encephalitis virus.

3. The RNA replicon according to claim 1, wherein the target gene comprises at least one of a tumor-specific or associated antigen, a pathogen-specific or associated antigen, a cytokine or a receptor thereof, a chemokine or a receptor thereof, a growth factor or a receptor thereof, an antibody protein, a bispecific antibody protein, a cytokine-antibody fusion protein, and an immune checkpoint-associated protein; preferably, the cytokine is GM-CSF, IFN-γ, IL-2, IL-12, or IL-15.

4. The RNA replicon according to claim 1, wherein a method for the mutation is PCR site-directed mutagenesis; and preferably, primers for the PCR site-directed mutagenesis comprise:
G357C F:
5′-GAAAATGAAGGAGCTCGCCGCCGTCATGAGCGACCC-3′;
G357C R:
5′-GCTCATGACGGCGGCGAGCTCCTTCATTTTCTTGTCC-3′;
G1569A/A1572C/C1575T F:
5′-GGAGCCCACTCTGGAAGCCGATGTCGACTTGATGTTACAAGAGG-
3′;
G1569A/A1572C/C1575T R:
5′-TAACATCAAGTCGACATCGGCTTCCAGAGTGGGCTCCTCAACATC-
3′;
A3821T F:
5′-CATTGGTGCTATAGCGCGGCTGTTCAAGTTTTCCCGGGTATGCAAA
C-3′:
T7VEESmaI R:
5′-GCTTAAGTTAGTTGCGGCCGCCCGGGTCGACTCTAG-3′;
T3922C F:
5′-GCCCGTACGCACAATCCTTACAAGCTTTCATCAAC-3′;
T3922C R:
5′-TGAAAGCTTGTAAGGATTGTGCGTACGGGCCTTG-3′;
G3892C F
5′-CTGTTTGTATTCATTCGGTACGATCGCAAGGCCCGTAC-3′;
G3892C R
5′-CCTTGCGATCGTACCGAATGAATACAAACAGAACTTC-3′;
A4714G F
5′-TATATCCTCGGAGAAGGCATGAGCAGTATTAGGTCG-3′;
A4714G R
5′-TAATACTGCTCATGCCTTCTCCGAGGATATACATGC-3′.

5. A vector, comprising the RNA replicon of claim 1.

6. A cell, comprising the vector of claim 5.

7. Use of the RNA replicon of claim 1 in any of (I)-(V):
(I) delivery of a target gene;
(II) achieving long-acting expression of a target gene;
(III) improving an expression quantity of a target gene;
(IV) gene therapy; and
(V) vaccine research and development.

8. A composition, comprising the RNA replicon of claim 1 or a vector comprising the RNA replicon of claim 1.

9. The composition according to claim 8, wherein the composition further comprises at least one of a medicinal diluent, a medicinal excipient, a medicinal vector, and a medicinal carrier; preferably, the composition further comprises other agents for combined use; the agents comprise but are not limited to: monoclonal antibody drugs, bispecific antibody drugs, antibody conjugates, fusion protein drugs, nucleic acid drugs, chemical drugs, blood product drugs, lipid drugs, or traditional Chinese medicine extracts;
preferably, the medicinal vector is a transfection reagent that is based on a cationic lipid and commercially available, a nonviral vector, a polymeric membrane, a biomimic membrane, a biological membrane, nanocarrier or a viral vector;
the transfection reagent comprises but is not limited to Lipofectamine2000, Lipofectamine3000, Lipofectamine8000, Lipofectamine LTX, Lipofectamine RNAiMAX, Lipofectamine MessengerMAX, or Invivofectamine 3.0;
the nonviral vector comprises but is not limited to a cationic polymer, a cationic liposome, an anionic liposome, a micelle, an inorganic nanoparticle, or a microsphere;
the polymeric membrane, the biomimic membrane, or the biological membrane comprises but is not limited to cytomembrane, an exosome or an extracellular vesicle; the viral vector comprises but is not limited to an adenovirus vector, a retrovirus, a lentivirus, a herpes virus, or a virus-like particle;
preferably, the nanocarrier comprises but is not limited to a polycationic peptide, a cationic lipid, an anionic lipid, a neutral lipid, a helper lipid, or an amphiphilic compound; and
preferably, the nanocarrier has a particle size of 20-350 nm and an electric charge of −40 mV to 50 mV.

10. A method for expressing a target gene in an organism, comprising following steps: administering to the organism the RNA replicon of claim 1 or a vector comprising the RNA replicon of claim 1; preferably, the organism being a procaryotic organism or a eukaryotic organism.

11. A vector, comprising the RNA replicon of claim 2.

12. A vector, comprising the RNA replicon of claim 3.

13. A vector, comprising the RNA replicon of claim 4.

14. Use of the RNA replicon of claim 2 in any of (I)-(V):
(I) delivery of a target gene;
(II) achieving long-acting expression of a target gene;
(III) improving an expression quantity of a target gene;
(IV) gene therapy; and
(V) vaccine research and development.

15. Use of the RNA replicon of claim 3 in any of (I)-(V):
(I) delivery of a target gene;
(II) achieving long-acting expression of a target gene;
(III) improving an expression quantity of a target gene;
(IV) gene therapy; and
(V) vaccine research and development.

16. Use of the RNA replicon of claim 4 in any of (I)-(V):
(I) delivery of a target gene;
(II) achieving long-acting expression of a target gene;
(III) improving an expression quantity of a target gene;
(IV) gene therapy; and
(V) vaccine research and development.

17. A composition, comprising the RNA replicon of claim 2 or a vector comprising the RNA replicon of claim 2.

18. A composition, comprising the RNA replicon of claim 3 or a vector comprising the RNA replicon of claim 3.

19. A composition, comprising the RNA replicon of claim 4 or a vector comprising the RNA replicon of claim 4.

20. A method for expressing a target gene in an organism, comprising following steps: administering to the organism the RNA replicon of claim 2 or a vector comprising the RNA replicon of claim 2; preferably, the organism being a procaryotic organism or a eukaryotic organism.


SEQUENCE LISTING (25 sequences)

SEQ ID NO: 1 - DNA, 7482 bp, synthetic construct
  source 1..7482: mol_type = other DNA
atggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaa

SEQ ID NO: 2 - DNA, 39 bp, synthetic construct
  source 1..39: mol_type = other DNA
aaaagcgcagtcaccaaaaaagatctagtggtgagcgcc

SEQ ID NO: 3 - DNA, 32 bp, synthetic construct
  source 1..32: mol_type = other DNA
atcgatgctgagggcgcgcccatatgctagac

SEQ ID NO: 4 - DNA, 35 bp, synthetic construct
  source 1..35: mol_type = other DNA
gcccgtacgcacaatccttacaagctttcatcaac

SEQ ID NO: 5 - DNA, 34 bp, synthetic construct
  source 1..34: mol_type = other DNA
tgaaagcttgtaaggattgtgcgtacgggccttg

SEQ ID NO: 6 - DNA, 38 bp, synthetic construct
  source 1..38: mol_type = other DNA
ctgtttgtattcattcggtacgatcgcaaggcccgtac

SEQ ID NO: 7 - DNA, 37 bp, synthetic construct
  source 1..37: mol_type = other DNA
ccttgcgatcgtaccgaatgaatacaaacagaacttc

SEQ ID NO: 8 - DNA, 36 bp, synthetic construct
  source 1..36: mol_type = other DNA
tatatcctcggagaaggcatgagcagtattaggtcg

SEQ ID NO: 9 - DNA, 36 bp, synthetic construct
  source 1..36: mol_type = other DNA
taatactgctcatgccttctccgaggatatacatgc

SEQ ID NO: 10 - DNA, 47 bp, synthetic construct
  source 1..47: mol_type = other DNA
cattggtgctatagcgcggctgttcaagttttcccgggtatgcaaac

SEQ ID NO: 11 - DNA, 36 bp, synthetic construct
  source 1..36: mol_type = other DNA
gcttaagttagttgcggccgcccgggtcgactctag

SEQ ID NO: 12 - DNA, 50 bp, synthetic construct
  source 1..50: mol_type = other DNA
aaaaaaaaaaaaaaaaaaaacgcgtcgaggggaattaattcttgaagacg

SEQ ID NO: 13 - DNA, 50 bp, synthetic construct
  source 1..50: mol_type = other DNA
ctttcttggcgctcaccactagatcttttttggtgactgcgcttttaatg

SEQ ID NO: 14 - DNA, 36 bp, synthetic construct
  source 1..36: mol_type = other DNA
gaaaatgaaggagctcgccgccgtcatgagcgaccc

SEQ ID NO: 15 - DNA, 37 bp, synthetic construct
  source 1..37: mol_type = other DNA
gctcatgacggcggcgagctccttcattttcttgtcc

SEQ ID NO: 16 - DNA, 44 bp, synthetic construct
  source 1..44: mol_type = other DNA
ggagcccactctggaagccgatgtcgacttgatgttacaagagg

SEQ ID NO: 17 - DNA, 45 bp, synthetic construct
  source 1..45: mol_type = other DNA
taacatcaagtcgacatcggcttccagagtgggctcctcaacatc

SEQ ID NO: 18 - DNA, 68 bp, synthetic construct
  source 1..68: mol_type = other DNA
gttctgtttgtattcattcggtacgatcgcaaggcccgtacgcacaatccttacaagctttcatcaac

SEQ ID NO: 19 - DNA, 67 bp, synthetic construct
  source 1..67: mol_type = other DNA
ttgatgaaagcttgtaaggattgtgcgtacgggccttgcgatcgtaccgaatgaatacaaacagaac

SEQ ID NO: 20 - DNA, 44 bp, synthetic construct
  source 1..44: mol_type = other DNA
gtctagtccgccaagtctagcatatggccaccatgtggctgcag

SEQ ID NO: 21 - DNA, 53 bp, synthetic construct
  source 1..53: mol_type = other DNA
aaaataaaaattttaaggcggcatgccaatcgccgcgagttctatgtaagcag

SEQ ID NO: 22 - DNA, 50 bp, synthetic construct
  source 1..50: mol_type = other DNA
gtctagtccgccaagtctagcatatggccaccatgaacgctacacactgc

SEQ ID NO: 23 - DNA, 48 bp, synthetic construct
  source 1..48: mol_type = other DNA
gtctagtccgccaagtctagcatatggccaccatggagacagacacac

SEQ ID NO: 24 - DNA, 32 bp, synthetic construct
  source 1..32: mol_type = other DNA
gtctagtccgccaagtctagcatatggccacc

SEQ ID NO: 25 - DNA, 11522 bp, synthetic construct
  source 1..11522: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctagcatatgggcgcgccctcagcatcgattcaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagtctagagtcgacccgggcggccgcaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagaattggcaagctgcttacatagaactcgcggcgattggcatgccgccttaaaatttttattttattttttcttttcttttccgaatcggattttgtttttaatatttcaaaaaaaaaaaaaaaaaaaaaaaaaacgcgtcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctctaatacgactcactatag